(19) United States
(12) Patent Application Publication
(10) Pub. No.: US 2014/0127171 A1
(43) Pub. Date: May 8, 2014

(54) TREATMENT OF CHRONIC POST-TRAUMATIC ENCEPHALOPATHY

(71) Applicants: Roger Nocera, Paradise Valley, AZ (US); James Eaker, Apoka, FL (US)

(72) Inventors: Roger Nocera, Paradise Valley, AZ (US); James Eaker, Apoka, FL (US)

(21) Appl. No.: 14/071,575

(22) Filed: Nov. 4, 2013

Related U.S. Application Data
(60) Provisional application No. 61/722,121, filed on Nov. 2, 2012.

(51) Int. Cl. A61K 35/50; A61K 41/00; A61N 5/06; A61K 45/06

(52) U.S. Cl. CPC A61K 35/50; A61K 45/06; A61K 41/00; A61N 5/062
USPC 424/93.7; 514/679; 514/458; 514/456; 514/560; 514/733; 424/682; 514/355; 514/251

(57) ABSTRACT

Methods of treating chronic post-traumatic encephalopathy (PTE) using regenerative approaches is described. In one embodiment, molecules with capability of stimulating endogenous neural stem cells is provided. In another embodiment, cell therapeutics are provided capable of addressing angiogenic deficits in patients suffering from PTE. In another embodiment, cells are utilized to induce activation of endogenous progenitor cells in the central nervous system of PTE patients. Furthermore, low level laser irradiation is disclosed as a means of treatment of PTE either through direct administration to CNS tissue for stimulation of endogenous progenitor cells and reparative processes, or together with administration of exogenous stem cells, whether autologous or allogeneic. In a further embodiment exogenous stem cells are pretreated with laser prior to administration.

### CROSS-REFERENCE TO RELATED APPLICATIONS

This application claims priority to U.S. Provisional Application No. 61/722,121, filed on Nov. 2, 2012, which is expressly incorporated by reference herein in its entirety.

### FIELD OF THE INVENTION

The invention pertains to the area of neural regeneration, more specifically, the invention pertains to stimulation of neuroregeneration as a means of treating patients with post traumatic encephalopathy, more specifically, the invention provides cells, protocols, chemicals, and treatment methods of ameliorating and reversing post traumatic encephalopathy.

### BACKGROUND

Injury to central nervous system (CNS) induced by acute insults including trauma, hypoxia and ischemia (caused by stroke or blunt force trauma) can affect both grey and white matter dependent on nature and severity. Injury to CNS involves neuro-inflammation. For example, leukocyte infiltration in the CNS after trauma or inflammation is triggered in part by up-regulation of the MCP-1 chemokine in astrocytes. Injury is often self-perpetuated by inflammatory mediators released that in turn activate toll like receptors [1]. Trauma is an injury or damage of the nerve. It may be spinal cord trauma, which is damage to the spinal cord that affects all nervous functions that are controlled at and below the level of the injury, including muscle control and sensation, or brain trauma, such as trauma caused by closed head injury. Cerebral hypoxia is a lack of oxygen specifically to the cerebral hemispheres, and more typically the term is used to refer to a lack of oxygen to the entire brain. Depending on the severity of the hypoxia, symptoms may range from confusion to irreversible brain damage, coma and death. Stroke is usually caused by reduced blood flow (ischemia) of the brain. It is also called cerebrovascular disease or accident. It is a group of brain disorders involving loss of brain functions that occurs when the blood supply to any part of the brain is interrupted.

Chronic traumatic encephalopathy (CTE), otherwise referred to as post traumatic encephalopathy, has been defined as a progressive neurodegenerative disease caused by repetitive head trauma. CTE was first described in 1928 when Dr. Harrison Martland, began to note a plethora of symptoms in boxers. In an article he published in the Journal of the American Medical Association entitled Punch Drunk, he describes the boxers, “cuckoo,” “goofy,” “cutting paper dolls,” or “slug nutty”. Punch drunk was later termed dementia pugilistica, literally meaning dementia of a fighter. However, with the evolution of sports like American football, these symptoms were also being reported in athletes other than boxers and was renamed chronic traumatic encephalopathy in the 1960s.

Autopsy results from soldiers and athletes have suggested a link between these emotional, cognitive, and physical manifestations and CTE. Mild traumatic brain injury (mTBI) is one of the most common neurologic disorders accounting for approximately 90% of all brain injuries sustained. Such injuries are a common occurrence in athletes with an estimated 1.6-3.8 million sport-related concussion annually in the USA. This can be seen as a gross underrepresentation of the true number as many athletes do not seek medical attention or vocalize their symptoms. This may be due to head trauma being regarded as benign, or in some the injury is not recognized at all. It was reported that each year more than 1.5 million Americans have mTBI with no loss of consciousness and no need for hospitalization as well as an equal number with conscious impairing trauma but insufficiently severe to require long-term hospitalization.

The brain requires about 20% of the circulation of blood in the body. The primary blood supply to the brain is through 2 arteries in the neck (the carotid arteries), which then branch off within the brain to multiple arteries that each supply a specific area of the brain. Even a temporary interruption to the blood flow can cause decreases in brain function (neurological deficit). The symptoms vary with the area of the brain affected and commonly include such problems as changes in vision (occipital lobe), speech changes (Broca' s Area), decreased movement or sensation in a part of the body (cerebellum), or changes in the level of consciousness (temporal lobe). If the blood flow is decreased for longer than a few seconds, brain cells in the area are destroyed (infarcted) causing permanent damage to that area of the brain or even death.

Stroke affects about 4 out of 1,000 people. It is the 3rd leading cause of death in most developed countries, including the U.S. The incidence of stroke rises dramatically with age, with the risk doubling with each decade after age 35. About 5% of people over age 65 have had at least one stroke. The disorder occurs in men more often than women. Causes of ischemic strokes are blood clots that form in the brain (thrombus) and blood clots or pieces of atherosclerotic plaque or other material that travel to the brain from another location (emboli). Bleeding (hemorrhage) within the brain may cause symptoms that mimic stroke. Strokes secondary to atherosclerosis (cerebral thrombosis) and strokes caused by embolism (moving blood clot) are the most common strokes. Traumatic nerve injury may concern both the CNS or the PNS. Traumatic brain injury, also simply called head injury or closed head injury, refers to an injury where there is damage to the brain because of an external blow to the head. It mostly happens during car or bicycle accidents, but may also occur as the result of near drowning, heart attack, stroke and infections. In military combat TBI is of particular relevance according to a recent report, between 2000 to 2010, there have been 178,876 cases of TBI (The Defense and Veterans Brain Injury Center, http://www.dvbic.org/TBI-Numbers.aspx). In the civilian population, according to the CDC there are between 1.5 and 2 million Americans that suffer TBI every year (“Traumatic Brain Injury,” Center for Disease Control and Prevention, National Center for Injury Prevention and Control, 2003, Vol. 2003). In the same reported, it is stated that TBI is responsible for 50,000 deaths and 100,000 hospitalizations annually. Additionally, over 80,000 individuals are disabled annually, approximately 17,000 of whom require specialized care for life. This type of traumatic brain injury would usually result due to the lack of oxygen or blood supply to the brain, and therefore can be referred to as an “anoxic injury”. Brain injury or closed head injury occurs when there is a blow to the head as in a motor vehicle accident or a fall. There may be a period of unconsciousness immediately following the trauma, which may last minutes, weeks or months. Primary brain damage occurs at the time of injury, mainly at the sites of impact, in particular when a skull fraction is present. Post traumatic encephalopathy possesses numerous similarities at the molecular and cellular level with stroke.

Large contusions may be associated with an intracerebral haemorrhage, or accompanied by cortical lacerations. Diffuse axonal injuries occur as a result of shearing and tensile strains of neuronal processes produced by rotational movements of the brain within the skull. There may be small heamorrhagic lesions or diffuse damage to axons, which can only be detected microscopically. Secondary brain damage occurs as a result of complications developing after the moment of injury. They include intracranial hemorrhage, traumatic damage to extracerebral arteries, intracranial herniation, hypoxic brain damage or meningitis.

Intracerebral hemorrhage (ICH) is a devastating type of stroke with high mortality and morbidity rate, effects 40 000 people in the United States annually [2]. Additionally, as stated above, ICH may be the result of blunt force trauma [3]. Despite of significant progress in diagnostics of this disease and on-going research on new therapy strategies there is no cure significantly decreasing mortality and improving life quality of survivals. Only fifty percent of patients will survive ICH, and these individuals will be afflicted with significant brain atrophy and lifelong neurological deficits[4]. Primary bleeding initiates several pathophysiological pathways leading to the secondary brain injury. This pathways include activation of microglia[5], production of pro-inflammatory cytokines[6] and reactive oxygen species[7] and infiltration by systemic immune cells[8]. Inflammation and ROS production are known events leading to disruption of blood-brain barrier, development of brain edema, the most live-threatened event after ICH. The long-term effect of factors mentioned above is a brain atrophy and long-lasting neurological deficit [9, 10].

Recent publications demonstrated that the brain after ICH is capable to self-repair [11]. It was also demonstrated that some endogenous and exogenous factors can stimulate post-injury neurogenesis and angiogenesis and promote brain recovery after ICH resulting in significant improvement of neurological outcome [12, 13].

Intracerebral hemorrhage is one of the most deadly kind of stoke accounting for approximately 5 to 15% of all types of stroke. ICH is more than twice more common as subarachnoid hemorrhage (SAH) and results in more disability and death than SAH or ischemic stroke. Despite of significant progress in diagnostics of this disease and on-going research on new therapy strategies there is no cure significantly decreasing mortality and improving life quality of survivals. The continuum of injury, whether by blunt force trauma, or cerebrovascular accident, all culminates into ischemic damage. Unfortunately, to date, with exception of thrombolytics, all clinical trials in stroke have failed. Agents tested included including antioxidants, calcium channel blockers, glutamate receptor blockers, and neurotrophic factors in over 1000 clinical trials [14].

The wars in Iraq and Afghanistan have produced a signature combat injury called post traumatic encephalopathy (PTE) caused by a primitive weapon, the improvised explosive device (IED), which has been ubiquitous on these two battlefields in the past decade. The classic PTE syndrome consists of chronic memory loss, refractory clinical depression, loss of brain executive function leading to loss of anger control producing an uncharacteristic propensity to inappropriate violence. Full blown PTE is essentially a life-sentence of psychogenic, self inflicted mental torture and progressive destruction of relationships until the attendant suffering is no longer bearable and PTE syndrome often ends with the injured soldier's suicide. Suicides are surging among America's troops, averaging nearly one a day this year—the fastest pace in the nation's decade of war. The suicide rate among the nation's active-duty military personnel has spiked this year, eclipsing the number of troops dying in battle and on pace to set a record annual high since the start of the wars in Iraq and Afghanistan more than a decade ago.

The USA professional sports community, a huge and growing American demographic, which includes our high school children, collage kids, as well as professional athletes is publicly reeling from their own tragic and growing PTE epidemic. They too desperately need a diagnostic method to detect the biochemical signature of PTE so that the team physicians can know when to remove an injured player with a fragile PTE brain from the field to prevent the disaster of advanced, untreatable and permanent PTE.

One of the key issues confounding medical efforts to deal effectively with PTE, besides having no effective treatment available, is that until now, we cannot reliably diagnose PTE so that doctors could know to immediately remove the injured soldier with PTE from the battlefield or the athlete from the playing field. This critical medical decision is made exquisitely difficult for a number of reasons. The injured soldier or athlete with PTE brain chemistry alterations loses some brain executive function, anger control and memory loss, but these dysfunctions, are very difficult to detect in the short term, before the soldier's life or athlete's life has devolved into a downward spiral of events caused by his PTE condition. Also, it is difficult for a military doctor or sports physician to measure inappropriate violent behavior and depression in a war zone or a violent sport. PTE by current medical practice standards can only be realistically and reliably identified with either a brain tissue biopsy, which is not a reasonable diagnostic option, or a brain tissue postmortem examination, which means a life has already been lost.

There is a need for the USA medical community to devise a practical diagnostic methodology to accurately and consistently identify those soldiers and athletes with PTE chemical alterations after the first brain insult, which currently cannot be detected with any combination of clinical data available, including all the sophisticated high tech diagnostic tools that we currently have in our medical armamentarium; not PET scans, CAT scans, nor MRI scans. Accordingly, there is a major need in the field for new approaches towards brain insults including PTE.

### SUMMARY OF THE INVENTION

Preferred embodiments are directed to the following methods. A method of treating post-traumatic encephalopathy (PTE) comprising of administering a patient in need of treatment a therapeutically sufficient concentration of cells with regenerative potential. The method, wherein said cells are selected from a group of cells comprising: a) type 2 monocytes; b) reprogrammed cells; c) hematopoietic stem cells; d) mesenchymal stem cells; e) endothelial progenitor cells; f) very small embryonic-like cells and g) a stem cell. The method wherein said type 2 monocytes are characterized by expression of the enzyme arginase. The method of claim 2, wherein said type 2 monocytes are generated by exposing monocytes to a type 2 cytokine. The method, wherein said type 4 cytokines are selected from a group comprising of: a) IL4; b) IL-10; c) TGF-beta; and e) VEGF.

The method, wherein said type 2 monocytes are generated by exposure to Substance P. The method, wherein said type 2 monocytes are derived from stromal vascular fraction of adipose tissue.

The method, wherein said reprogrammed cells are selected from a group comprising of: a) therapeutic cells exposed to cytoplasm of a cell possessing a more immature phenotype than said target cell; b) cells are induced to dedifferentiate through the administration of a chemical agent; c) cells that are induced to dedifferentiate through transfection with genes capable of inducing dedifferentiation; and d) cells that are created as a result of a fusion with a more undifferentiated cell.

The method, wherein said therapeutic cells exposed to cytoplasm of a cell possessing a more immature phenotype than said target cell are fibroblasts transfected with cytoplasm from a group of cells comprising of: a) embryonic stem cells; b) inducible pluripotent cells; and e) fetal stem cells. The method, wherein said therapeutic cells are further treated with an agent selected from: a) a histone deacetylase inhibitor; and b) a DNA methyltransferase inhibitor. The method, wherein said reprogrammed cell is an induced pluripotent stem cell. The method, wherein said induced pluripotent cells is a cell transfected with genes selected from a group comprising of: OCT4, SOX2, NANOG, and KLF-4.

The method, wherein said chemical agent capable of inducing dedifferentiation is selected from a group of agents comprising of: valproic acid, 5-azacytidine, and trichostatin A. The method wherein said reprogrammed cell is generated through fusing a fibroblast with an embryonic stem cell. The method, wherein said reprogrammed cell is generated through fusing a fibroblast with a parthenogenic stem cell. The method, wherein said hematopoietic stem cells are cells expressing markers selected from a group comprising of: a) CD34; b) CD117; c) aldehyde dehydrogenase; d) CD45; and e) CD133. The method wherein said hematopoietic stem cells do not express substantial levels of markers selected from a group comprising of: a) CD14; b) CD38; and c) CD56.

The method, wherein said hematopoietic stem cells are capable of causing formation of cells derived from the myeloid, lymphoid and erythroid lineage. The method, wherein said mesenchymal stem cells are characterized by expression of markers selected from a group comprising of: CD90, CD105, CD73, and Stro-1. The method, wherein said mesenchymal stem cells lack significant expression of the markers CD14, CD34, and CD45. The method, wherein said endothelial progenitor cells are characterized by an agent capable of bind a molecule selected from the group of: a) CD34; b) CD133; c) KDR-1; and d) CD166. The method wherein said endothelial progenitor cells are capable of forming endothelial colonies when plated in a methylcellulose culture dish. The method, wherein said very small embryonic like cells are less than 7 microns in diameter. The method, wherein said cells express a molecule selected from a group comprising of: a) wnt-5; b) CD34; c) CD133; d) Oct-4; e) Nanog; and f) SSEA-1. The method, wherein said stem cells are selected from a group comprising of: embryonic stem cells, cord blood stem cells, placental stem cells, bone marrow stem cells, amniotic fluid stem cells, neuronal stem cells, circulating peripheral blood stem cells, germinal stem cells, adipose tissue derived stem cells, exfoliated teeth derived stem cells, hair follicle stem cells, dermal stem cells, parthenogenically derived stem cells, reprogrammed stem cells and side population stem cells.

The method, wherein said embryonic stem cells are totipotent. The method, wherein said embryonic stem cells express one or more antigens selected from a group consisting of: stage-specific embryonic antigens (SSEA) 3, SSEA 4, Tra-1-60 and Tra-1-81, Oct-3/4, Cripto, gastrin-releasing peptide (GRP) receptor, podocalyxin-like protein (PODXL), Rex-1, GCTM-2, Nanog, and human telomerase reverse transcriptase (hTERT).

The method, wherein said cord blood stem cells are multipotent and capable of differentiating into endothelial, smooth muscle, and neuronal cells. The method, wherein said cord blood stem cells are identified based on expression of one or more antigens selected from a group comprising: SSEA-3, SSEA-4, CD9, CD34, c-kit, OCT-4, Nanog, and CXCR-4. The method, wherein said cord blood stem cells do not express one or more markers selected from a group comprising of: CD3, CD34, CD45, and CD11b. The method, wherein said placental stem cells are isolated from the placental structure. The method, wherein said placental stem cells are identified based on expression of one or more antigens selected from a group comprising: Oct-4, Rex-1, CD9, CD13, CD29, CD44, CD166, CD90, CD105, SH-3, SH-4, TRA-1-60, TRA-1-81, SSEA-4 and Sox-2.

The method, wherein said bone marrow stem cells comprise of bone marrow mononuclear cells. The method, wherein said bone marrow stem cells are enriched for expression of CD133. The method, wherein said amniotic fluid stem cells are isolated by introduction of a fluid extraction means into the amniotic cavity under ultrasound guidance. The method, wherein said amniotic fluid stem cells are selected based on expression of one or more of the following antigens: SSEA3, SSEA4, Tra-1-60, Tra-1-81, Tra-2-54, HLA class I, CD13, CD44, CD49b, CD105, Oct-4, Rex-1, DAZL and Runx-1. The method, wherein said amniotic fluid stem cells are selected based on lack of expression of one or more of the following antigens: CD34, CD45, and HLA Class II.

The method, wherein said neuronal stem cells are selected based on expression of one or more of the following antigens: RC-2, 3CB2, BLB, Sox-2hh, GLAST, Pax 6, nestin, Muashi-1, NCAM, A2B5 and prominin. The method, wherein said circulating peripheral blood stem cells are characterized by ability to proliferate in vitro for a period of over 3 months. The method, wherein said circulating peripheral blood stem cells are characterized by expression of CD34, CXCR4, CD117, CD113, and c-met. The method, wherein said circulating peripheral blood stem cells lack substantial expression of differentiation associated markers.

The method, wherein said differentiation associated markers are selected from a group comprising of CD2, CD3, CD4, CD11, CD11a, Mac-1, CD14, CD16, CD19, CD24, CD33, CD36, CD38, CD45, CD56, CD64, CD68, CD86, CD66b, and HLA-DR. The method, wherein said mesenchymal stem cells express one or more of the following markers: STRO-1, CD105, CD54, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, and THY-1. The method, wherein said mesenchymal stem cells do not express substantial levels of HLA-DR, CD117, and CD45.

The method, wherein said germinal stem cells express markers selected from a group comprising of: Oct4, Nanog, Dppa5 Rbm, cyclin A2, Tex18, Stra8, Dazl, beta1- and alpha6-integrins, Vasa, Fragilis, Nobox, c-Kit, Sca-1 and Rex1. The method, wherein said adipose tissue derived stem cells express markers selected from a group comprising of: CD13, CD29, CD44, CD63, CD73, CD90, CD166, Aldehyde dehydrogenase (ALDH), and ABCG2. The method, wherein said adipose tissue derived stem cells are a population of purified mononuclear cells extracted from adipose tissue capable of proliferating in culture for more than 1 month. The method, wherein said exfoliated teeth derived stem cells express markers selected from a group comprising of: STRO-1, CD146 (MUC18), alkaline phosphatase, MEPE, and bFGF. The method, wherein said hair follicle stem cells express markers selected from a group comprising of: cytokeratin 15, Nanog, and Oct-4. The method, wherein said hair follicle stem cells are capable of proliferating in culture for a period of at least one month. The method, wherein said hair follicle stem cells secrete one or more of the following proteins when grown in culture: basic fibroblast growth factor (bFGF), endothelin-1 (ET-1) and stem cell factor (SCF). The method, wherein said dermal stem cells express markers selected from a group comprising of: CD44, CD13, CD29, CD90, and CD105. The method, wherein said dermal stem cells are capable of proliferating in culture for a period of at least one month. The method, wherein said parthenogenically derived stem cells are generated by addition of a calcium flux inducing agent to activate an oocyte followed by enrichment of cells expressing markers selected from a group comprising of SSEA-4, TRA 1-60 and TRA 1-81.

The method, wherein said reprogrammed stem cells are selected from a group comprising of: cells subsequent to a nuclear transfer, cells subsequent to a cytoplasmic transfer, cells treated with a DNA methyltransferase inhibitor, cells treated with a histone deacetylase inhibitor, cells treated with a GSK-3 inhibitor, cells induced to dedifferentiate by alteration of extracellular conditions, and cells treated with various combination of the mentioned treatment conditions. The method, wherein said nuclear transfer comprises introducing nuclear material to a cell substantially enucleated, said nuclear material deriving from a host whose genetic profile is sought to be dedifferentiated. The method, wherein said cytoplasmic transfer comprises introducing cytoplasm of a cell with a dedifferentiated phenotype into a cell with a differentiated phenotype, such that said cell with a differentiated phenotype substantially reverts to a dedifferentiated phenotype.

The method, wherein said DNA demethylating agent is selected from a group comprising of: 5-azacytidine, psammaplin A, and zebularine. The method, wherein said histone deacetylase inhibitor is selected from a group comprising of: valproic acid, trichostatin-A, trapoxin A and depsipeptide. The method, wherein said cells are identified based on expression multidrug resistance transport protein (ABCG2) or ability to efflux intracellular dyes such as rhodamine-123 and or Hoechst 33342. The method, wherein said cells are derived from tissues such as pancreatic tissue, liver tissue, smooth muscle tissue, striated muscle tissue, cardiac muscle tissue, bone tissue, bone marrow tissue, bone spongy tissue, cartilage tissue, liver tissue, pancreas tissue, pancreatic ductal tissue, spleen tissue, thymus tissue, Peyer's patch tissue, lymph nodes tissue, thyroid tissue, epidermis tissue, dermis tissue, subcutaneous tissue, heart tissue, lung tissue, vascular tissue, endothelial tissue, blood cells, bladder tissue, kidney tissue, digestive tract tissue, esophagus tissue, stomach tissue, small intestine tissue, large intestine tissue, adipose tissue, uterus tissue, eye tissue, lung tissue, testicular tissue, ovarian tissue, prostate tissue, connective tissue, endocrine tissue, and mesentery tissue.

The method, wherein said committed progenitor cells are selected from a group comprising of: endothelial progenitor cells, neuronal progenitor cells, and hematopoietic progenitor cells. The method, wherein said committed endothelial progenitor cells are purified from the bone marrow. The method, wherein said committed endothelial progenitor cells are purified from peripheral blood. The method, wherein said committed endothelial progenitor cells are purified from peripheral blood of a patient whose committed endothelial progenitor cells are mobilized by administration of a mobilizing agent or therapy. The method, wherein said mobilizing agent is selected from a group comprising of: G-CSF, M-CSF, GM-CSF, 5-FU, IL-1, IL-3, kit-L, VEGF, Flt-3 ligand, PDGF, EGF, FGF-1, FGF-2, TPO, IL-11, IGF-1, MGDF, NGF, HMG CoA) reductase inhibitors and small molecule antagonists of SDF-1. The method, wherein said mobilization therapy is selected from a group comprising of: exercise, hyperbaric oxygen, autohemotherapy by ex vivo ozonation of peripheral blood, and induction of SDF-1 secretion in an anatomical area outside of the bone marrow. The method, wherein said committed endothelial progenitor cells express markers selected from a group comprising of: CD31, CD34, AC133, CD146 and flk1. The method, wherein said committed hematopoietic cells are purified from the bone marrow.

The method, wherein said committed hematopoietic progenitor cells are purified from peripheral blood. The method, wherein said committed hematopoietic progenitor cells are purified from peripheral blood of a patient whose committed hematopoietic progenitor cells are mobilized by administration of a mobilizing agent or therapy. The method, wherein said mobilizing agent is selected from a group comprising of: G-CSF, M-CSF, GM-CSF, 5-FU, IL-1, IL-3, kit-L, VEGF, Flt-3 ligand, PDGF, EGF, FGF-1, FGF-2, TPO, IL-11, IGF-1, MGDF, NGF, HMG CoA) reductase inhibitors and small molecule antagonists of SDF-1. The method, wherein said mobilization therapy is selected from a group comprising of: exercise, hyperbaric oxygen, autohemotherapy by ex vivo ozonation of peripheral blood, and induction of SDF-1 secretion in an anatomical area outside of the bone marrow. The method, wherein said committed hematopoietic progenitor cells express the marker CD133. The method, wherein said committed hematopoietic progenitor cells express the marker CD34.

The method, wherein an antioxidant is administered at a therapeutically sufficient concentration to a patient in need thereof. The method, wherein said antioxidant is selected from a group comprising of: ascorbic acid and derivatives thereof, alpha tocopherol and derivatives thereof, rutin, quercetin, hesperedin, lycopene, resveratrol, tetrahydrocurcumin, rosmarinic acid, Ellagic acid, chlorogenic acid, oleuropein, alpha-lipoic acid, glutathione, polyphenols, pycnogenol. The method, wherein said laser irradiation is performed on the central nervous system prior to administration of said cells with regenerative potential.

The method, wherein said laser irradiation is provided on the skin surface. The method, wherein said laser irradiation is provided transcranially. The method, wherein said laser irradiation is provided in at least one wavelength, said wavelength in a range between about 620 nanometers and about 1070 nanometers. The method, wherein said laser irradiation is provided for a sufficient time and energy intensity to augment activity of said administered cells containing a concentrated stem cell population. The method, wherein said activity of said administered cells is selected from a group comprising of: a) enhanced cytokine production; b) enhanced ability to differentiate into cells of the pulmonary architecture; c) augmented ability to produce antiapoptic factors to cells of pulmonary architecture; d) increased angiogenic activity; and e) inhibition of inflammatory cytokine production. The method wherein said laser irradiation is administered by a light source between approximately 100 .mu.W/cm.sup.2 to approximately 10 W/cm.sup.2. The method, wherein light energy having a wavelength in the visible to near-infrared wavelength range and a predetermined power density to the brain of the subject is administered in a non-invasive manner comprises transmitting light energy through the scalp and the skull to the brain, wherein the predetermined power density is at least about 0.01 mW/cm.sup.2 at a depth of approximately 2 centimeters below the dura.

The method, wherein said predetermined power density is selected from the range of about 0.01 mW/cm.sup.2 to about 100 mW/cm.sup.2 at a depth of approximately 2 centimeters below the dura. The method wherein said predetermined power density is selected from the range of about 0.01 mW/cm.sup.2 to about 15 mW/cm.sup.2 at a depth of approximately 2 centimeters below the dura. The method, wherein said light energy has a wavelength of about 630 nm to about 904 nm. The method wherein the light energy has a wavelength of about 780 nm to about 840 nm. The method wherein delivering a neurologic function enhancing effective amount of light energy to the brain comprises placing a light source in contact with a region of skin adjacent to the brain. The method, wherein delivering light energy comprises determining a surface power density of the light energy sufficient to deliver the predetermined power density of light energy to the brain. The method wherein determining a surface power density of the light energy sufficient to deliver the predetermined power density of light energy to the brain consists of identifying the surface power density of the light energy sufficient for the light energy to traverse the distance between the skin surface and the brain. The method wherein determining the surface power density further comprises determining the surface power density sufficient to penetrate the skull. The method, wherein the treatment proceeds for a period of about 30 seconds to about 2 hours. The method, wherein the light energy is pulsed. The method wherein said light energy is administered via a laser. The method, wherein said light energy is administered through the utilization of a light emitting diode.

The method, wherein said cells possessing regenerative ability are exposed to light in a manner to augment therapeutic activities of said cells. The method, wherein said cells possessing regenerative ability are exposed to light in a manner to augment cytokine production activities of said cells. The method, wherein said cells possessing regenerative ability are exposed to light in a manner to augment growth factor production activities of said cells. The method, wherein said cells possessing regenerative ability are exposed to light in a manner to augment angiogenic factor production activities of said cells. The method, wherein said cells possessing regenerative ability are exposed to light in a manner to augment migratory activities of said cells. The method, wherein said migratory activities of said cells comprises ability to home to a site of tissue injury.

A method for treatment of post-traumatic encephalopathy providing: a) a cell with ability to inhibit host inflammatory reactions; b) an agent or therapy capable of mobilizing endogenous stem cells; and c) administration of said cell capable of inhibiting host inflammatory reactions prior to, subsequent to, or concurrently with said agent or therapy capable of mobilizing endogenous stem cells. The method, wherein said cell with ability to inhibit host inflammatory reactions is selected from a group consisting of: a) a mesenchymal stem cell; b) an alternatively activated macrophage; c) a myeloid suppressor cell; and d) an immature dendritic cell. The method, wherein said cell with ability to inhibit host inflammatory reactions is autologous to said host. The method, wherein said cell with ability to inhibit host inflammatory reactions are allogeneic to said host. The method, wherein said cell with ability to inhibit host inflammatory reactions are peripheral blood derived mesenchymal stem cells. The method, wherein said agent capable of mobilizing endogenous stem cells is selected from a group comprising of: M-CSF, G-CSF, GM-CSF, an antagonist of CXCR-4, an antagonist of VLA-4, fucoidan, IVIG, parathyroid hormone, and cyclophosphamide.

The method, wherein said treatment capable of mobilizing endogenous stem cells is selected from a group of treatments consisting of: hyperbaric oxygen, exercise, and autohemotherapy using extracorporeal ozonation. A method of treating post traumatic encephalopathy comprising of: a) obtaining an agent with ability to inhibit host inflammatory reactions; b) obtaining an agent or therapy capable of mobilizing endogenous stem cells; and c) administration of said agent with ability to inhibit host inflammatory reactions prior to, subsequent to, or concurrently with said agent or therapy capable of mobilizing endogenous stem cells. The method, wherein said agent with ability to inhibit host inflammatory reactions is selected from a group consisting of: a) a small molecule; b) a nucleic acid; c) a protein.

The method, wherein inhibition of said host inflammatory reactions is defined as downregulation of inflammation in the CNS of a patient. The method, wherein inflammation is defined as increased local numbers of immunological cells, and/or increased activity of immunological cells as compared to a healthy aged matched control. The method, wherein inflammation is defined as production of soluble mediators in comparison to a healthy age matched control selected from a group comprising of: TNF-alpha, TNF-beta, IL-1, IL-2, IL-6, IL-12, IL-18, IL-21, IL-23, interferon alpha, interferon beta, interferon gamma, interferon tau, and prostaglandin E2.

The method, wherein said anti-inflammatory small molecule agent is selected from a group comprising of: pioglitazone, aspirin, ibuprofen, n-acetylcysteine, and resveratrol. The method, wherein said agent capable of mobilizing endogenous stem cells is selected from a group comprising of: M-CSF, G-CSF, GM-CSF, an antagonist of CXCR-4, an antagonist of VLA-4, fucoidan, parathyroid hormone, IVIG, and cyclophosphamide. The method, wherein said treatment capable of mobilizing endogenous stem cells is selected from a group of treatments consisting of: hyperbaric oxygen, exercise, and autohemotherapy using extracorporeal ozonation. The method, wherein said agent capable of stimulating proliferation of stem cells is selected from one or more agents of a group comprising of: prolactin; growth hormone, estrogen, ciliary neurotrophic factor (CNTF), pituitary adenylate cyclase activating polypeptide (PACAP), fibroblast growth factor (FGF), transforming growth factor alpha (TGF.alpha.), epidermal growth factor (EGF), erythropoietin, human chorionic gonadotrophin, cardiotrophin, IGF, thalidomide, valproic acid, G-CSF, trichostatin A, sodium phenylbutyrate, 5-azacytidine, and FSH.

### DETAILED DESCRIPTION OF THE ILLUSTRATED EMBODIMENTS

Embodiments of the present invention are described below. It is, however, expressly noted that the present invention is not limited to these embodiments, but rather the intention is that modifications that are apparent to the person skilled in the art and equivalents thereof are also included.

The invention provides means of treating PTE through administration of various stem cells, activators of stem cells, and laser means of enhancing stem cell activation. The underlying theme of the invention teaches the use of cells with stem cell-like properties for the treatment of PTE. Specific properties of stem cells that are suitable for use in practicing the current invention are: a) ability to both increase endothelial function, as well as induce neoangiogenesis; b) ability to prevent atrophy, as well as to differentiate into functional neuronal tissue; and c) ability to induce local resident stem/progenitor cells to proliferate through secretion of soluble factors, as well as via membrane bound activities. In one embodiment of the invention, stem cells are collected from an autologous patient, expanded ex vivo, and reintroduced into said patient at a concentration and frequency sufficient to cause therapeutic benefit in PTE. Said stem cells are selected for ability to cause: neoangiogenesis, prevention of tissue atrophy, and regeneration of functional tissue. Stem cells chosen may be selected from a group comprising of: embryonic stem cells, cord blood stem cells, placental stem cells, bone marrow stem cells, amniotic fluid stem cells, neuronal stem cells, circulating peripheral blood stem cells, mesenchymal stem cells, germinal stem cells, adipose tissue derived stem cells, exfoliated teeth derived stem cells, hair follicle stem cells, dermal stem cells, parthenogenically derived stem cells, reprogrammed stem cells and side population stem cells.

Selection of Stem Cells for PTE Therapy: When selecting stem cells for use in the practice of the current invention, several factors must be taken into consideration, such as: ability for ex vivo expansion without loss of therapeutic activity, ease of extraction, general potency of activity, and potential for adverse effects. Ex vivo expansion ability of stem cells can be measured using typical proliferation and colony assays known to one skilled in the art, while identification of therapeutic activity depends on functional assays that test biological activities such as: ability to support endothelial function, ability to protect neurons from degeneration/atrophy, and, ability to inhibit smooth muscle atrophy/degeneration. Assessment of therapeutic activity can also be performed using surrogate assays which detect markers associated with a specific therapeutic activity. Such markers include CD34 or CD133, which are associated with stem cell activity and ability to support angiogenesis [15]. Other assays useful for identifying therapeutic activity of stem cell populations for use with the current invention include evaluation of production of factors associated with the therapeutic activity desired. For example, identification and quantification of production of FGF, VEGF, angiopoietin, or other such angiogenic molecules may be used to serve as a guide for approximating therapeutic activity in vivo [16]. Additionally, secretion of factors that inhibit smooth muscle atrophy or neuronal dysfunction may also be used as a marker for identification of cells that are useful for practicing the current invention.

In another embodiment of the invention cord blood stem cells are administered systemically into a patient suffering from PTE. Said cord blood stem cells may be administered as a heterogenous population of cells by the administration of cord blood mononuclear cells. Said cells may be isolated according to many methods known in the art. In one particular method, cord blood is collected from fresh placenta and mononuclear cells are purified by centrifugation using a density gradient such as Ficoll or Percoll, in another method cord blood mononuclear cells are isolated from contaminating erythrocytes and granulocytes by the Hetastarch with a 6% (wt/vol) hydroxyethyl starch gradient. Cells are subsequently washed to remove contaminating debris, assessed for viability, and administered at a concentration and frequency sufficient to induce therapeutic benefit.

In another embodiment of the invention, cord blood stem cells are fractionated and the fraction with enhanced therapeutic activity is administered to the patient. Enrichment of cells with therapeutic activity may be performed using physical differences, electrical potential differences, differences in uptake or excretion of certain compounds, as well as differences in expression marker proteins. Distinct physical property differences between stem cells with high proliferative potential and low proliferative potential are known. Accordingly, in some embodiments of the invention, it will be useful to select cord blood stem cells with a higher proliferative ability, whereas in other situations, a lower proliferative ability may be desired. In some embodiments of the invention, cells are directly injected into the area of need, such as intrathecally, in which case it will be desirable for said stem cells to be substantially differentiated, whereas in other embodiments, cells will be administered systemically and it this case with may be desirable for the administered cells to be less differentiated, so has to still possess homing activity to the area of need. In embodiments of the invention where specific cellular physical properties are the basis of differentiating between cord blood stem cells with various biological activities, discrimination on the basis of physical properties can be performed using a Fluorescent Activated Cell Sorter (FACS), through manipulation of the forward scatter and side scatter settings. Other methods of separating cells based on physical properties include the use of filters with specific size ranges, as well as density gradients and pheresis techniques. When differentiation is desired based on electrical properties of cells, techniques such as electrophotoluminescence may be used in combination with a cell sorting means such as FACS. Selection of cells based on ability to uptake certain compounds can be performed using, for example, the ALDESORT system, which provides a fluorescent-based means of purifying cells with high aldehyde dehydrogenase activity. Cells with high levels of this enzyme are known to possess higher proliferative and self-renewal activities in comparison to cells possessing lower levels. Other methods of identifying cells with high proliferative activity includes identifying cells with ability to selectively efflux certain dyes such as rhodamine-123 and or Hoechst 33342. Without being bound to theory, cells possessing this property often express the multidrug resistance transport protein ABCG2, and are known for enhanced regenerative ability compared to cells which do not possess this efflux mechanism. In other embodiments cord blood cells are purified for certain therapeutic properties based on expression of markers. In one particular embodiment, cord blood cells are purified for the phenotype of endothelial precursor cells. Said precursors, or progenitor cells express markers such as CD133, and/or CD34. Said progenitors may be purified by positive or negative selection using techniques such as magnetic activated cell sorting (MACS), affinity columns, FACS, panning, or by other means known in the art. Cord blood derived endothelial progenitor cells may be administered directly into the target tissue for PTE, or may be administered systemically. Another variation of this embodiment is the use of differentiation of said endothelial precursor cells in vitro, followed by infusion into a patient. Verification for endothelial differentiation may be performed by assessing ability of cells to bind FITC-labeled Ulex europaeus agglutinin-1, ability to endocytose acetylated Di-LDL, and the expression of endothelial cell markers such as PECAM-1, VEGFR-2, or CD31.

Certain desired activities can be endowed onto said cord blood stem cells prior to administration into the patient. In one specific embodiment cord blood cells may be “activated” ex vivo by a brief culture in hypoxic conditions in order to upregulate nuclear translocation of the HIF-1 transcription factor and endow said cord blood cells with enhanced angiogenic potential. Hypoxia may be achieved by culture of cells in conditions of 0.1% oxygen to 10% oxygen, preferably between 0.5% oxygen and 5% oxygen, and more preferably around 1% oxygen. Cells may be cultured for a variety of timepoints ranging from 1 hour to 72 hours, more preferably from 13 hours to 59 hours and more preferably around 48 hours. Assessment of angiogenic, and other desired activities useful for the practice of the current invention, can be performed prior to administration of said cord blood cells into the patient. Assessment methods are known in the art and include measurement of angiogenic factors, ability to support viability and activity of cells associated with neural function, as well as ability to induce regeneration of said cellular components associated with PTE.

In addition to induction of hypoxia, other therapeutic properties can be endowed unto cord blood stem cells through treatment ex vivo with factors such as dedifferentiating compounds, proliferation inducing compounds, or compounds known to endow and/or enhance cord blood cells to possess properties useful for the practice of the current invention. In one embodiment cord blood cells are cultured with an inhibitor of the enzyme GSK-3 in order to enhance expansion of cells with pluripotent characteristics while not increasing the rate of differentiation. In another embodiment, cord blood cells are cultured in the presence of a DNA methyltransferase inhibitor such as 5-azacytidine in order to endow a “de-differentiation” effect. In another embodiment cord blood cells are cultured in the presence of a differentiation agent that skews said cord blood stem cells to generate enhance numbers of cells which are useful for treatment of PTE after said cord blood cells are administered into a patient

In contrast to cord blood stem cells, placental stem cells may be purified directly from placental tissues, said tissues including the chorion, amnion, and villous stroma [17, 18]. In another embodiment of the invention, placental tissue is mechanically degraded in a sterile manner and treated with enzymes to allow dissociation of the cells from the extracellular matrix. Such enzymes include, but not restricted to trypsin, chymotrypsin, collagenases, elastase and/or hylauronidase. Suspension of placental cells are subsequently washed, assessed for viability, and may either be used directly for the practice of the invention by administration either locally or systemically. Alternatively, cells may be purified for certain populations with increased biological activity. Purification may be performed using means known in the art, and described above for purification of cord blood stem cells, or may be achieved by positive selection for the following markers: SSEA3, SSEA4, TRA1-60, TRA1-81, c-kit, and Thy-1. In some situations it will be desirable to expand cells before introduction into the human body. Expansion can be performed by culture ex vivo with specific growth factors [19, 20]. The various embodiments of the invention described above for cord blood and embryonic stem cells can also be applied for placental stem cells.

Bone marrow stem cells may be used either freshly isolated, purified, or subsequent to ex vivo culture. A typical bone marrow harvest for collecting starting material for practicing one embodiment of the invention involves a bone marrow harvest with the goal of acquiring approximately 5-700 ml of bone marrow aspirate. Numerous techniques for the aspiration of marrow are described in the art and part of standard medical practice. One particular methodology that may be attractive due to decreased invasiveness is the “mini-bone marrow harvest” [21]. Said aspirate is used as a starting material for purification of cells with PTE inhibiting activity. In one specific embodiment bone marrow mononuclear cells are isolated by pheresis or gradient centrifugation. Numerous methods of separating mononuclear cells from bone marrow are known in the art and include density gradients such as Ficoll Histopaque at a density of approximately 1.077 g/ml or Percoll gradient. Separation of cells by density gradients is usually performed by centrifugation at approximately 450 g for approximately 25-60 minutes. Cells may subsequently be washed to remove debris and unwanted materials. Said washing step may be performed in phosphate buffered saline at physiological pH. An alternative method for purification of mononuclear cells involves the use of apheresis apparatus such as the CS3000-Plus blood-cell separator (Baxter, Deerfield, USA), the Haemonetics separator (Braintree, Mass.), or the Fresenius AS 104 and the Fresenius AS TEC 104 (Fresenius, Bad Homburg, Germany) separators. In addition to injection of mononuclear cells, purified bone marrow subpopulations may be used. Additionally, ex vivo expansion and/or selection may also be utilized for augmentation of desired biological properties for use in treatment of PTE. The various embodiments of the invention described above for cord blood and embryonic stem cells can also be applied for bone marrow stem cells.

Amniotic fluid is routinely collected during amniocentesis procedures. One method of practicing the current invention is utilizing amniotic fluid derived stem cells for treatment of PTE. In one embodiment amniotic fluid mononuclear cells are utilized therapeutically in an unpurified manner. Said amniotic fluid stem cells are administered either locally or systemically in a patient suffering from ED. In other embodiments amniotic fluid stem cells are substantially purified based on expression of markers such as SSEA-3, SSEA4, Tra-1-60, Tra-1-81 and Tra-2-54, and subsequently administered. In other embodiments cells are cultured, as described in U.S. patent application # 20050054093, expanded, and subsequently infused into the patient. Amniotic stem cells are described in the following references [22-24]. One particular aspect of amniotic stem cells that makes them amenable for use in practicing certain aspects of the current invention is their bi-phenotypic profile as being both mesenchymal and neural progenitors [25]. This property is useful for treatment of patients with PTE in which neural regeneration is required to a greater extent than vascular repair. The various embodiments of the invention described above for cord blood and embryonic stem cells can also be applied for amniotic fluid stem cells.

Stem cells committed to the neuronal lineage, or neuronal progenitor cells, are used in the practice of some specific embodiments of the invention. Said cells may be generated by differentiation of embryonic stem cells, may be freshly isolated from fetal tissue (ie mesencephalic), may be generated by transdifferentiation, or by reprogramming of a cell. Neuronal progenitors are selected by use of markers such as polysialyated N-CAM, N-CAM, A2B5, nestin and vimentin. The various embodiments of the invention described above for cord blood and embryonic stem cells can also be applied for neuronal stem cells.

A wide variety of stem cells are known to circulate in the periphery. These include multipotent, pluripotent, and committed stem cells. In some embodiments of the invention mobilization of stem cells is induced in order to increase the number of circulating stem cells, so that harvesting efficiency is increased. Said mobilization allows for harvest of cells with desired properties for practice of the invention without the need to perform bone marrow puncture. A variety of methods to induce mobilization are known. Methods such as administration of cytotoxic chemotherapy, for example, cyclophosphamide or 5-fluoruracil are effective but not preferred in the context of the current invention due to relatively unacceptable adverse events profile. Suitable agents useful for mobilization include: granulocyte colony stimulating factor (G-CSF), granulocyte-macrophage colony stimulating factor (GM-CSF), interleukin 1 (IL-1), interleukin 3 (IL-3), stem cell factor (SCF, also known as steel factor or kit ligand), vascular endothelial growth factor (VEGF), Flt-3 ligand, platelet-derived growth factor (PDGF), epidermal growth factor (EGF), fibroblast growth factor-1 (FGF-1), fibroblast growth factor-2 (FGF-2), thrombopoietin (TPO), interleukin-11 (IL-11), insulin-like growth factor-1 (IGF-1), megakaryocyte growth and development factor (MGDF), nerve growth factor (NGF), hyperbaric oxygen, and 3-hydroxy-3-methyl glutaryl coenzyme A (HMG CoA)reductase inhibitors. The various embodiments of the invention described above for cord blood and embryonic stem cells can also be applied for circulating peripheral blood stem cells.

In a preferred embodiment, donors (either autologous or allegeneic) are mobilized by administration of G-CSF (filgrastim: neupogen) at a concentration of 10 ug/kg/day by subcutaneous injection for 2-7 days, more preferably 4-5 days. Peripheral blood mononuclear cells are collected using an apheresis device such as the AS 104 cell separator (Fresenius Medical). 1-40×109 mononuclear cells are collected, concentrated and injected intrathecally. Alternatively, cells may be injected systemically, or in an area proximal to the area of brain injury. Identification of such occlusion is routinely known in the art, as well as identification of the area of fibrosis. Variations of this procedure may include steps such as subsequent culture of cells to enrich for various populations known to possess angiogenic and/or neurogenic, and/or anti-atrophy Additionally cells may be purified for specific subtypes before and/or after culture. Treatments can be made to the cells during culture or at specific timepoints during ex vivo culture but before infusion in order to generate and/or expand specific subtypes and/or functional properties.

In one embodiment mesenchymal cells are generated through culture. For example, U.S. Pat. No. 5,486,359 describes methods for culturing such and expanding mesenchymal stem cells, as well as providing antibodies for use in detection and isolation. U.S. Pat. No. 5,942,225 teaches culture techniques and additives for differentiation of such stem cells which can be used in the context of the present invention to produce increased numbers of cells with angiogenic capability. Although U.S. Pat. No. 6,387,369 teaches use of mesenchymal stem cells for regeneration of cardiac tissue, we believe that in accordance with published literature [26, 27] stem cells generated through these means are actually angiogenically potent and therefore may be utilized in the context of the current invention for treatment/amelioration of PTE. Without being bound to a specific theory or mechanism of action, it appears that mesenchymal stem cells induce angiogenesis through production of factors such as vascular endothelial growth factor, hepatocyte growth factor, adrenomedullin, and insulin-like growth factor-1 [28].

Mesenchymal stem cells are classically obtained from bone marrow sources for clinical use, although this source may have disadvantages because of the invasiveness of the donation procedure and the reported decline in number of bone marrow derived mesenchymal stem cells during aging. Alternative sources of mesenchymal stem cells include adipose tissue [29], placenta [18, 30], scalp tissue [31] and cord blood [32]. A recent study compared mesenchymal stem cells from bone marrow, cord blood and adipose tissue in terms of colony formation activity, expansion potential and immunophenotype. It was demonstrated that all three sources produced mesenchymal stem cells with similar morphology and phenotype. Ability to induce colony formation was highest using stem cells from adipose tissue and interestingly in contrast to bone marrow and adipose derived mesenchymal cells, only the cord blood derived cells lacked ability to undergo adipocyte differentiation. Proliferative potential was the highest with cord blood mesenchymal stem cells which were capable of expansion to approximately 20 times, whereas cord blood cells expanded an average of 8 times and bone marrow derived cells expanded 5 times [33]. Accordingly, one skilled in the art will understand that mesenchymal stem cells for use with the present invention may be selected upon individual patient characteristics and the end result sought. For example, if autologous mesenchymal stem cells are available in the form of adipocyte-derived cells, it will be useful to utilize this source instead of allogeneic cord-blood derived cells. Alternatively, cord blood derived mesenchymal stem cells may be more advantageous for use in situations where autologous cells are not available, and expansion is sought.

The ability of mesenchymal stem cells from the cord blood to expand in vitro also allows the possibility of genetically modifying these cells in order to: a) decrease immunogeneicity; b) enhance angiogenic potential; and c) augment survival following administration. However it should be noted that such ex vivo manipulation is applicable to all cell types described in the current application.

In situations where a decrease in immunogenicity is sought, cells may be transfected using immune suppressive agents. Said agents include soluble factors, membrane-bound factors, and enzymes capable of causing localized immune suppression. Examples of soluble immune suppressive factors include: IL-4 [34], IL-10 [35], IL-13 [36], TGF-b [37], soluble TNF-receptor [38], and IL-1 receptor agonist [39]. Membrane-bound immunoinhibitor molecules that may be transfected into stem cells for use in practicing the current invention include: HLA-G [40], FasL [41], PD-1L [42], Decay Accelerating Factor [43], and membrane-associated TGF-b [44]. Enzymes which may be transfected in order to cause localized immune suppression include indolamine 2,3 dioxygenase [45] and arginase type II [46]. In order to optimize desired immune suppressive ability, a wide variety of assays are known in the art, including mixed lymphocyte culture, ability to generate T regulatory cells in vitro, and ability to inhibit natural killer or CD8 cell cytotoxicity.

In situations where increased angiogenic potential of said mesenchymal stem cells is desired, mesenchymal stem cells may be transfected with genes such as VEGF[47], FGF1 [48], FGF2 [49], FGF4 [50], FrzA [51], and angiopoietin [52]. Ability to induce angiogenesis may be assessed in vitro prior to administration of said transfected cells in vivo. Methods of assessing in vitro angiogenesis stimulating ability are well known in the art and include measuring proliferation of human umbilical vein derived endothelial cells.

Since one of the problems of cell therapy in general is viability of the infused cells subsequent to administration, it may be desired in some forms of the invention to transfect mesenchymal cells with genes protecting said cells from apoptosis. Anti-apoptotic genes suitable for transfection may include bc1-2 [53], bc1-x1 [54], and members of the XIAP family [55]. Alternatively it may be desired to increase the proliferative lifespan of said mesenchymal stem cells through transfection with enzymes associated with anti-senescence activity. Said enzymes may include telomerase or histone deacetylases. The various embodiments of the invention described above for cord blood and embryonic stem cells can also be applied for mesenchymal stem cells.

Adipose derived stem cells express markers such as CD9; CD29 (integrin beta 1); CD44 (hyaluronate receptor); CD49d,e (integrin alpha 4, 5); CD55 (decay accelerating factor); CD105 (endoglin); CD106 (VCAM-1); CD166 (ALCAM). These markers are useful not only for identification but may be used as a means of positive selection, before and/or after culture in order to increase purity of the desired cell population. In terms of purification and isolation, devices are known to those skilled in the art for rapid extraction and purification of cells adipose tissues. U.S. Pat. No. 6,316,247 describes a device which purifies mononuclear adipose derived stem cells in an enclosed environment without the need for setting up a GMP/GTP cell processing laboratory so that patients may be treated in a wide variety of settings. One embodiment of the invention involves attaining 10-200 ml of raw lipoaspirate, washing said lipoaspirate in phosphate buffered saline, digesting said lipoaspirate with 0.075% collagenase type I for 30-60 min at 37° C. with gentle agitation, neutralizing said collagenase with DMEM or other medium containing autologous serum, preferably at a concentration of 10% v/v, centrifuging the treated lipoaspirate at approximately 700-2000 g for 5-15 minutes, followed by resuspension of said cells in an appropriate medium such as DMEM. Cells are subsequently filtered using a cell strainer, for example a 100 μm nylon cell strainer in order to remove debris. Filtered cells are subsequently centrifuged again at approximately 700-2000 g for 5-15 minutes and resuspended at a concentration of approximately 1×106/cm2 into culture flasks or similar vessels. After 10-20 hours of culture non-adherent cells are removed by washing with PBS and remaining cells are cultured at similar conditions as described above for culture of cord blood derived mesenchymal stem cells. Upon reaching a concentration desired for clinical use, cells are harvested, assessed for purity and administered in a patient in need thereof as described above. The various embodiments of the invention described above for cord blood and embryonic stem cells can also be applied for adipose derived stem cells.

Deciduous teeth (baby teeth) have been recently identified as a source of pluripotent stem cells with ability to differentiate into endothelial, neural, and bone structures. Said pluripotent stem cells have been termed “stem cells from human exfoliated deciduous teeth” (SHED). One of the embodiments of the current invention involves utilization of this novel source of stem cells for the treatment of PTE. In one embodiment of the invention, SHED cells are administered systemically or locally into a patient with PTE at a concentration and frequency sufficient for induction of therapeutic effect. SHED cells can be purified and used directly, certain sub-populations may be concentrated, or cells may be expanded ex vivo under distinct culture conditions in order to generate phenotypes desired for maximum therapeutic effect. Growth and expansion of SHED has been previously described by others. In one particular method, exfoliated human deciduous teeth are collected from 7- to 8-year-old children, with the pulp extracted and digested with a digestive enzyme such as collagenase type I. Concentrations necessary for digestion are known and may be, for example 1-5 mg/ml, or preferable around 3 mg/ml. Additionally dispase may also be used alone or in combination, concentrations of dispase may be 1-10 mg/ml, preferably around 4 mg/ml. Said digestion is allowed to occur for approximately 1 h at 37° C. Cells are subsequently washed and may be used directly, purified, or expanded in tissue culture. The various embodiments of the invention described above for cord blood and embryonic stem cells can also be applied for exfoliated teeth stem cells.

The bulge area of the hair follicle bulge is an easily accessible source of pluripotent mesenchymal-like stem cells. One embodiment of the current invention is the use of hair follicle stem cells for treatment of PTE. Said cells may be used therapeutically once freshly isolated, or may be purified for particular sub-populations, or may be expanded ex vivo prior to use. Purification of hair follicle stem cells may be performed from cadavers, from healthy volunteers, or from patients undergoing plastic surgery. Upon extraction, scalp specimens are rinsed in a wash solution such as phosphate buffered saline or Hanks and cut into sections 0.2-0.8 cm. Subcutaneous tissue is de-aggregated into a single cell suspension by use of enzymes such as dispase and/or collagenase. In one variant, scalp samples are incubated with 0.5% dispase for a period of 15 hours. Subsequently, the dermal sheath is further enzymatically de-aggregated with enzymes such as collagenase D. Digestion of the stalk of the dermal papilla, the source of stem cells is confirmed by visual microscopy. Single cell suspensions are then treated with media containing fetal calf serum, and concentrated by pelletting using centrifugation. Cells may be further purified for expression of markers such as CD34, which are associated with enhanced proliferative ability. In one embodiment of the invention, collected hair follicle stem cells are induced to differentiate in vitro into neural-like cells through culture in media containing factors such as FGF-1, FGF-2, NGF, neurotrophin-2, and/or BDNF. Confirmation of neural differentiation may be performed by assessment of markers such as Muhashi, polysialyated N-CAM, N-CAM, A2B5, nestin, vimentin glutamate, synaptophysin, glutamic acid decarboxylase, serotonin, tyrosine hydroxylase, and GABA. Said neuronal cells may be administered systemically, or locally in a patient with ED. The various embodiments of the invention described above for cord blood and embryonic stem cells can also be applied for hair follicle stem cells.

Parthenogenically derived stem cells can be generated by addition of a calcium flux inducing agent to activate oocytes, followed by purifying and expanding cells expressing embryonic stem cell markers such as SSEA-4, TRA 1-60 and/or TRA 1-81. Said parthenogenically derived stem cells are totipotent and can be used in a manner similar to that described for embryonic stem cells in the practice of the current invention. The various embodiments of the invention described above for cord blood and embryonic stem cells can also be applied for parthenogenically derived stem cells.

Reprogramming of non-stem cells to endow them with stem cell characteristics can generate stem cells for use in the practice of the current invention. The advantage of reprogramming cells is that ability to withdraw autologous cells, which may have limited stem cell potential, endow said autologous cells with stem cell, or stem cell-like, properties, and reintroduce said autologous cells into the patient. The various embodiments of the invention described above for cord blood and embryonic stem cells can also be applied for reprogrammed stem cells.

Cells expressing the ability to efflux certain dyes, including but not limited to rhodamin-123 are associated with stem cell-like properties. Said cells can be purified from tissue subsequent to cell dissociation, based on efflux properties. Accordingly, in one embodiment of the current invention, tissue derived side population cells may be utilized either freshly isolated, sorted into subpopulations, or subsequent to ex vivo culture, for the treatment of PTE. For use in the invention, side population cells may be derived from tissues such as pancreatic tissue, liver tissue, smooth muscle tissue, striated muscle tissue, cardiac muscle tissue, bone tissue, bone marrow tissue, bone spongy tissue, cartilage tissue, liver tissue, pancreas tissue, pancreatic ductal tissue, spleen tissue, thymus tissue, Peyer's patch tissue, lymph nodes tissue, thyroid tissue, epidermis tissue, dermis tissue, subcutaneous tissue, heart tissue, lung tissue, vascular tissue, endothelial tissue, blood cells, bladder tissue, kidney tissue, digestive tract tissue, esophagus tissue, stomach tissue, small intestine tissue, large intestine tissue, adipose tissue, uterus tissue, eye tissue, lung tissue, testicular tissue, ovarian tissue, prostate tissue, connective tissue, endocrine tissue, and mesentery tissue. More optimally, side population cells obtained from smooth muscle tissue are administered at a concentration and frequency sufficient to induce a therapeutic effect on ED. The various embodiments of the invention described above for cord blood and embryonic stem cells can also be applied for side population stem cells.

In one embodiment of the invention bone marrow mononuclear cells are concentrated in an injection solution, which may be saline, mixtures of autologous plasma together with saline, or various concentrations of albumin with saline. Ideally pH of the injection solution is from about 6.4 to about 8.3, optimally 7.4. Excipients may be used to bring the solution to isotonicity such as, 4.5% mannitol or 0.9% sodium chloride, pH buffers with art-known buffer solutions, such as sodium phosphate. Other pharmaceutically acceptable agents can also be used to bring the solution to isotonicity, including, but not limited to, dextrose, boric acid, sodium tartrate, propylene glycol, polyols (such as mannitol and sorbitol) or other inorganic or organic solutes.

The current invention teaches the previously undisclosed combination of using low level laser irradiation to augment activity of autologous stem cells, or cell populations containing stem cells. It has been known in the art that low level laser irradiation has beneficial effects on growth factor production, including ability to stimulate cytokines such as VEGF [56], EGF [57], and PDGF [58], which have been demonstrated to act as antiapoptotic agents for neural cells, as well as to support angiogenesis. Additionally, direct induction of angiogenesis has been previously reported by administration of low level laser irradiation [59]. Although in vitro mesenchymal stem cell activation has been previously described by treatment with low level laser irradiation [60], to our knowledge, the combination of an autologous cell treatment with this approach has not been reported.

In one embodiment, the invention teaches the use of the stromal vascular fraction isolated from lipoaspirates together with low level laser irradiation for treatment of PTE. The cells may be treated ex vivo or treated in vivo. Administration of laser energy may be performed through the tissue, or conversely by use of fiber optics or endoscopic techniques. Without being bound to theory, the therapeutic effects of the stromal vascular fraction may be related to its various constituents. Specifically, it is known that alternatively activated macrophages, T regulatory cells, and mesenchymal stem cells [61] are found in this cell population. Within the context of the current invention, low level laser irradiation, is used to augment therapeutic properties of these cells for use in PTE, which is associated with immune hyperactivity/inflammation. Mesenchymal stem cells not only have the potential to immune modulate through induction of T regulatory cells [62-64], suppression of macrophage activation [65] and release of anti-inflammatory factors [66], but also have ability to induce regeneration of damaged alveolar tissue [67].

Advantages of the approach described in the current invention include: a) Ease of implementation. Stem cells do not require expansion in the current invention. Various stem cell therapies have commonly been performed using autologous administration of a heterogenous population of cells that are only partially purified for their stem cell contents. For example, administration of autologous bone marrow mononuclear cells, or stromal vascular fraction isolated from adipose tissue may be performed as a one day procedure without the need for expansion. In the context of the current invention, the efficacy of this simple procedure is augmented by administration of low level laser irradiation to the brain or CNS; b) Synergy between mechanical stem cell deposition and low level laser chemoattraction. Administration of various stem cell populations intrathecally results in deposition due to mechanical peculiarities. By administration of low level laser irradiation, the invention aims to increase retention of administered stem cells in the CNS; and c) Activation of cytokine/reparative properties of administered stem cells in situ. The invention seeks to augment the existing growth factor secreting properties of administered stem cells by causing activation once they have reached the tissue where intervention is necessary.

In one aspect of the invention, a liposuction is performed a patient with PTE to extract approximately 500-1000 ml of adipose tissue. Said adipose tissue may be processed for extraction of stromal vascular fraction cells according to methods well known in the art, as described, for example, in U.S. Pat. Nos. 6,153,432, 6,391,297, 6,429,013, 6,555,374, 6,841,150, 7,001,746 and 7,033,587 and incorporated by reference. A specific protocol for clinical isolation and administration of autologous stromal vascular fraction cells may be used as described [61]. Administration of stromal vascular fraction cells is performed at a concentration of 1-100 million cells per injection, with a preferable dose of 20-70 million cells, with a more preferable dose around 40 million cells. Frequency of administration may be performed based on patient response clinically, or based on biomarkers of stem cell activity, such as secretion of systemic growth factors, said growth factors include IGF-1, FGF-1, FGF-2, PDGF, EGF, VEGF, and KGF-1. Irradiation may be accomplished using several low level laser devices that are currently available on the market. Low level laser irradiation may be performed with at least one wavelength between 620 nanometers and about 1070 nanometers using a light source generating approximately 100 .mu.W/cm.sup.2 to approximately 10 W/cm.sup.2. Administration of low level laser irradiation also can use different combinations of wavelengths, both in specific wavelengths used and in the number of different wavelengths used, e.g., two, three or more different wavelengths. In some embodiments one can use one or several separate narrow bands (FWHM up to 50 nm) in combination with one or several broad bands (FWHM>50 nm). Additionally, various treatments can be combined where, for example, the treatments are found to by synergistic and/or when the efficacy of the treatments is not reduced when combined. Determination of laser approach may use tissue depth as an indicator. For example, wavelengths of 632 nm (He—Ne), 670 nm (InGaAlP), 810 nm/830 nm (GaAlAs), 850 nm/904 nm, LED (e.g., 660 nm) may be used. In one embodiment, the wavelength of 810 nm/830 nm is used based on penetration depth, while wavelengths of 632 nm (He—Ne) are known have lesser penetration capabilities. An example of a laser device is the Expanded Spectrum Photo Therapy Device 510k #K083580. Several patents and patent applications describe various low level irradiation approaches which are incorporated by reference, these include #20070219604, U.S. Pat. No. 6,471,716, 20090254154, 20090074754, 20090069872, 20070072824, U.S. Pat. Nos. 7,052,167, 7,125,416, and 6,702,837. It is apparent to one of skill in the art that modification and optimization may be accomplished based on several parameters which would serve to achieve the object of the invention, which is decreasing progression of PTE and inducing regeneration of neural tissue. Parameters of interest include: a) Enhancing cytokine production, wherein cytokines of interest are associated with protection from COPD. Said cytokines include IGF-1, VEGF, FGF-1, FGF-2, PDGF-1, and KGF-1; b) Reduction of profibrotic cytokines such as TGF-beta; c) inhibition of apoptosis of pulmonary cells; d) stimulation of angiogenic effects; and e) suppression of inflammatory cytokines.

Further embodiments herein are directed to the use of magnetic resonance spectroscopy (MRS) to detect PTE in patients. MRS is a noninvasive technique that analyzes proteins in the brain, can identify neurochemical changes that occur as a result of repetitive head injury. MR spectroscopy can be used to measure N-acetyl aspartate abnormalities in the brain, which is a chemical that is a measure of the number of functioning brain cells. A first concussive shockwave exposure creates microscopic brain damage, which alters brain chemistry leading to a supercharged physiological state researchers call “immuno-excito-toxicity” affecting important immune effector cells: microglia and sometimes astrocytes. If the head is again hit by another concussive blow, the state of immuno-excito-toxicity is exacerbated, possibly permanently (second hit phenomenon). At this point even if removed from all forms of trauma, this toxicity causes progressive autoimmune brain damage leading to accumulations of abnormal proteins like tau and amyloid, very similar those seen in Alzheimer's Disease brains.

It is this ongoing and progressive autoimmune reaction, “immuno-excito-toxicity” that MR spectroscopy can diagnose and for which our treatment regimen is aimed at eliminating. Because immuno-excito-toxicity is a biochemical event, it can be reversed with safe orally administered biochemicals called nutraceuticals as outlined in the peer review article, Blaylock RL, Maroon J. Natural plant products and extracts that reduce immunoexcitotoxicity-associated neurodegeneration and promote repair within the central nervous system. Surg Neurol Int 2012; 3:19

Once the immuno-excito-toxicity biochemical abnormalities are diagnosed with MRS we will treat the subjects with a regimen of anti-inflammatory nutraceuticals and/or laser and/or morsalized amniotic membrane derived cells formulated specifically to safely reverse the immuno-toxicity. Our success will be documented by a repeat MR spectroscopy exam after this treatment. With this methodology various combinations of nutraceuticals can be assessed for effectiveness. We will therefore establish the criteria for MRS diagnosis for PTE and simultaneously develop a safe and effective treatment for PTE and stop their autoimmune progressive brain tissue destruction so that soldiers and athletes can resume their respective activities in the field. Likewise without immuno-excito-toxicity these patients brain tissue is no longer vulnerable to the second hit phenomenon and are therefore safe to resume activity.

All references listed herein are expressly incorporated by reference in their entireties. The invention may be embodied in other specific forms besides and beyond those described herein. The foregoing embodiments are therefore to be considered in all respects illustrative rather than limiting, and the scope of the invention is defined and limited only by the appended claims and their equivalents, rather than by the foregoing description.

### REFERENCES

1. Arumugam, T. V., et al., Toll-like receptors in ischemia-reperfusion injury. Shock, 2009. 32 (1): p. 4-16.
2. Feigin, V. L., et al., Worldwide stroke incidence and early case fatality reported in 56 population-based studies: a systematic review. Lancet Neurol, 2009. 8 (4): p. 355-69.
3. Morrison, A. L., et al., Acceleration-deceleration injuries to the brain in blunt force trauma. The American journal of forensic medicine and pathology, 1998. 19 (2): p. 109-12.
4. Broderick, J. P., et al., Guidelines for the management of spontaneous intracerebral hemorrhage: A statement for healthcare professionals from a special writing group of the Stroke Council, American Heart Association. Stroke, 1999. 30 (4): p. 905-15.
5. Wang, J. and S. E. Tsirka, Tuftsin fragment 1-3 is beneficial when delivered after the induction of intracerebral hemorrhage. Stroke, 2005. 36 3): p. 613-8.
6. Hijioka, M., et al., Therapeutic effect of nicotine in a mouse model of intracerebral hemorrhage. J Pharmacol Exp Ther, 2011.
7. Gu, Y., C. M. Dee, and J. Shen, Interaction of free radicals, matrix metalloproteinases and caveolin-1 impacts blood-brain barrier permeability. Front Biosci (Schol Ed), 2011. 3: p. 1216-31.
8. Ma, Q., et al., Vascular adhesion protein-1 inhibition provides antiinflammatory protection after an intracerebral hemorrhagic stroke in mice. J Cereb Blood Flow Metab, 2011. 31 (3): p. 881-93.
9. Lekic, T., et al., Protective effect of melatonin upon neuropathology, striatal function, and memory ability after intracerebral hemorrhage in rats. J Neurotrauma, 2010. 27 (3): p. 627-37.
10. MacLellan, C. L., et al., Intracerebral hemorrhage models in rat: comparing collagenase to blood infusion. J Cereb Blood Flow Metab, 2008. 28 (3): p. 516-25.
11. Masuda, T., et al., Increase in neurogenesis and neuroblast migration after a small intracerebral hemorrhage in rats. Neurosci Lett, 2007. 425 (2): p. 114-9.
12. Hua, Y., et al., Thrombin and brain recovery after intracerebral hemorrhage. Stroke, 2009. 40 (3 Suppl): p. S88-9.
13. Yang, S., et al., Effects of thrombin on neurogenesis after intracerebral hemorrhage. Stroke, 2008. 39 (7): p. 2079-84.
14. Barreto, G., et al., Astrocytes: targets for neuroprotection in stroke. Central nervous system agents in medicinal chemistry, 2011. 11 (2): p. 164-73.
15. Higashi, Y., et al., Oxidative stress, endothelial function and angiogenesis induced by cell therapy and gene therapy. Curr Pharm Biotechnol, 2006. 7 (2): p. 109-16.
16. Tonnesen, M. G., X. Feng, and R. A. Clark, Angiogenesis in wound healing. J Investig Dermatol Symp Proc, 2000. 5 (1): p. 40-6.
17. Demir, R., et al., Classification of human placental stem villi: review of structural and functional aspects. Microsc Res Tech, 1997. 38 (1-2): p. 29-41.
18. Portmann-Lanz, C. B., et al., Placental mesenchymal stem cells as potential autologous graft for pre- and perinatal neuroregeneration. Am J Obstet Gynecol, 2006. 194 (3): p. 664-73.
19. Mohamed, A. A., et al., Ex vivo expansion of stem cells: defining optimum conditions using various cytokines. Lab Hematol, 2006. 12 (2): p. 86-93.
20. Kashiwakura, I. and T. A. Takahashi, Fibroblast growth factor and ex vivo expansion of hematopoietic progenitor cells. Leuk Lymphoma, 2005. 46 (3): p. 329-33.
21. Mineishi, S., [Immunobiology of mini-transplant]. Nippon Rinsho, 2003. 61 (9): p. 1489-94.
22. Bossolasco, P., et al., Molecular and phenotypic characterization of human amniotic fluid cells and their differentiation potential. Cell Res, 2006. 16 (4): p. 329-36.
23. Sartore, S., et al., Amniotic mesenchymal cells autotransplanted in a porcine model of cardiac ischemia do not differentiate to cardiogenic phenotypes. Eur J Cardiothorac Surg, 2005. 28 (5): p. 677-84.
24. Prusa, A. R., et al., Neurogenic cells in human amniotic fluid. Am J Obstet Gynecol, 2004. 191 (1): p. 309-14.
25. Tsai, M. S., et al., Clonal amniotic fluid-derived stem cells express characteristics of both mesenchymal and neural stem cells. Biol Reprod, 2006. 74 (3): p. 545-51.
26. Caplan, A. I. and J. E. Dennis, Mesenchymal stem cells as trophic mediators. J Cell Biochem, 2006.
27. Shyu, K. G., et al., Mesenchymal stem cells are superior to angiogenic growth factor genes for improving myocardial performance in the mouse model of acute myocardial infarction. J Biomed Sci, 2006. 13 (1): p. 47-58.
28. Nagaya, N., et al., Transplantation of mesenchymal stem cells improves cardiac function in a rat model of dilated cardiomyopathy. Circulation, 2005. 112 (8): p. 1128-35.
29. Knippenberg, M., et al., Adipose tissue-derived mesenchymal stem cells acquire bone cell-like responsiveness to fluid shear stress on osteogenic stimulation. Tissue Eng, 2005. 11 (11-12): p. 1780-8.
30. Zhang, X., et al., Mesenchymal progenitor cells derived from chorionic villi of human placenta for cartilage tissue engineering. Biochem Biophys Res Commun, 2006. 340 (3): p. 944-52.
31. Shih, D. T., et al., Isolation and characterization of neurogenic mesenchymal stem cells in human scalp tissue. Stem Cells, 2005. 23 (7): p. 1012-20.
32. Kadivar, M., et al., In vitro cardiomyogenic potential of human umbilical vein-derived mesenchymal stem cells. Biochem Biophys Res Commun, 2006. 340 (2): p. 639-4
33. Kern, S., et al., Comparative Analysis of Mesenchymal Stem Cells from Bone Marrow, Umbilical Cord Blood or Adipose Tissue. Stem Cells, 2006.
34. Jansen, J. H., et al., Interleukin-4. A regulatory protein. Blut, 1990. 60 (5): p. 269-74.
35. Zhou, X., et al., Boosting interleukin-10 production: therapeutic effects and mechanisms. Curr Drug Targets Immune Endocr Metabol Disord, 2005. 5 (4): p. 465-75.
36. Mentink-Kane, M. M. and T. A. Wynn, Opposing roles for IL-13 and IL-13 receptor alpha 2 in health and disease. Immunol Rev, 2004. 202: p. 191-202.
37. Kriegel, M. A., et al., Transforming growth factor-beta: recent advances on its role in immune tolerance. Curr Rheumatol Rep, 2006. 8 (2): p. 138-44.
38. Fernandez-Botran, R., F. A. Crespo, and X. Sun, Soluble cytokine receptors in biological therapy. Expert Opin Biol Ther, 2002. 2 (6): p. 585-605.
39. Dayer, J. M., Evidence for the biological modulation of IL-1 activity: the role of IL-1Ra. Clin Exp Rheumatol, 2002. 20 (5 Suppl 27): p. S14-20.
40. Rouas-Freiss, N., et al., HLA-G proteins in cancer: do they provide tumor cells with an escape mechanism? Cancer Res, 2005. 65 (22): p. 10139-44.
41. Bohana-Kashtan, O. and C. I. Civin, Fas ligand as a tool for immunosuppression and generation of immune tolerance. Stem Cells, 2004. 22 (6): p. 908-24.
42. Okazaki, T. and T. Honjo, The PD-1-PD-L pathway in immunological tolerance. Trends Immunol, 2006. 27 (4): p. 195-201.
43. Longhi, M. P., et al., Holding T cells in check—a new role for complement regulators? Trends Immunol, 2006. 27 (2): p. 102-8.
44. Wahl, S. M., J. M. Orenstein, and W. Chen, TGF-beta influences the life and death decisions of T lymphocytes. Cytokine Growth Factor Rev, 2000. 11 (1-2): p. 71-9.
45. Mellor, A. L. and D. H. Munn, IDO expression by dendritic cells: tolerance and tryptophan catabolism. Nat Rev Immunol, 2004. 4 (10): p. 762-74.
46. Serafini, P., I. Borrello, and V. Bronte, Myeloid suppressor cells in cancer: recruitment, phenotype, properties, and mechanisms of immune suppression. Semin Cancer Biol, 2006. 16 (1): p. 53-65.
47. Matsumoto, R., et al., Vascular endothelial growth factor-expressing mesenchymal stem cell transplantation for the treatment of acute myocardial infarction. Arterioscler Thromb Vasc Biol, 2005. 25 (6): p. 1168-73.
48. Klein, S., M. Roghani, and D. B. Rifkin, Fibroblast growth factors as angiogenesis factors: new insights into their mechanism of action. Exs, 1997. 79: p. 159-92.
49. Chen, C. H., et al., Fibroblast growth factor 2: from laboratory evidence to clinical application. Curr Vasc Pharmacol, 2004. 2 (1): p. 33-43.
50. Grines, C., et al., Angiogenic gene therapy with adenovirus 5 fibroblast growth factor-4 (Ad5FGF-4): a new option for the treatment of coronary artery disease. Am J Cardiol, 2003. 92 (9B): p. 24N-31N.
51. Dufourcq, P., et al., FrzA, a secreted frizzled related protein, induced angiogenic response. Circulation, 2002. 106 (24): p. 3097-103.
52. Morisada, T., et al., Angiopoietins and angiopoietin-like proteins in angiogenesis. Endothelium, 2006. 13 (2): p. 71-9.
53. Murphy, E., K. Imahashi, and C. Steenbergen, Bc1-2 regulation of mitochondrial energetics. Trends Cardiovasc Med, 2005. 15 (8): p. 283-90.
54. Harada, H. and S. Grant, Apoptosis regulators. Rev Clin Exp Hematol, 2003. 7 (2): p. 117-38.
55. Guegan, C., et al., PTD-XIAP protects against cerebral ischemia by anti-apoptotic and transcriptional regulatory mechanisms. Neurobiol Dis, 2006. 22 (1): p. 177-86.
56. Khanna, A., et al., Augmentation of the expression of proangiogenic genes in cardiomyocytes with low dose laser irradiation in vitro. Cardiovasc Radiat Med, 1999. 1 (3): p. 265-9.
57. Mvula, B., T. J. Moore, and H. Abrahamse, Effect of low-level laser irradiation and epidermal growth factor on adult human adipose-derived stem cells. Lasers Med Sci, 2009.
58. Safavi, S. M., et al., Effects of low-level He—Ne laser irradiation on the gene expression of IL-1beta, TNF-alpha, IFN-gamma, TGF-beta, bFGF, and PDGF in rat's gingiva. Lasers Med Sci, 2008. 23 (3): p. 331-5.
59. Franca, C. M., et al., Low-intensity red laser on the prevention and treatment of induced-oral mucositis in hamsters. J Photochem Photobiol B, 2009. 94 (1): p. 25-31.
60. Horvat-Karajz, K., et al., In vitro effect of carboplatin, cytarabine, paclitaxel, vincristine, and low-power laser irradiation on murine mesenchymal stem cells. Lasers Surg Med, 2009. 41 (6): p. 463-9.
61. Riordan, N. H., et al., Non-expanded adipose stromal vascular fraction cell therapy for multiple sclerosis. J Transl Med, 2009. 7: p. 29.
62. Madec, A. M., et al., Mesenchymal stem cells protect NOD mice from diabetes by inducing regulatory T cells. Diabetologia, 2009. 52 (7): p. 1391-9.
63. Gonzalez-Rey, E., et al., Human adipose-derived mesenchymal stem cells reduce inflammatory and T-cell responses and induce regulatory T cells in vitro in rheumatoid arthritis. Ann Rheum Dis, 2009.
64. Ye, Z., et al., Immunosuppressive effects of rat mesenchymal stem cells: involvement of CD4+CD25+ regulatory T cells. Hepatobiliary Pancreat Dis Int, 2008. 7 (6): p. 608-14.
65. Tsyb, A. F., et al., In vitro inhibitory effect of mesenchymal stem cells on zymosan-induced production of reactive oxygen species. Bull Exp Biol Med, 2008. 146 (1): p. 158-64.
66. Ortiz, L. A., et al., Interleukin 1 receptor antagonist mediates the antiinflammatory and antifibrotic effect of mesenchymal stem cells during lung injury. Proc Natl Acad Sci USA, 2007. 104 (26): p. 11002-7.
67. Mora, A. L. and M. Rojas, Aging and lung injury repair: a role for bone marrow derived mesenchymal stem cells. J Cell Biochem, 2008. 105 (3): p. 641-7.

1. A method of treating post traumatic encephalopathy (PTE) in a patient suspected of suffering from PTE comprising:
(a) detecting PTE and brain inflammation in a patient by observing abnormally high levels of choline and threonine and abnormally low levels of GABA and N-Acetyl Aspartate in the patient's brain matter using a first round of magnetic resonance spectroscopy (MRS);
(b) administering a pharmaceutical grade food nutraceutical in sufficient amount to ameliorate the inflammation in the brain; and
(c) performing a second round of MRS on said patient and comparing to the first round of MRS to confirm improvement in brain biochemical levels of choline, threonine, GABA, and N-Acetyl Aspartate to confirm amelioration of brain inflammation.

2. The method of claim 1 further comprising administering laser irradiation therapy to the patient between the first and second rounds of MRS.

3. The method of claim 1 further comprising administering amniotic membrane adult stem cells to the patient between the first and second rounds of MRS.

4. The method of claim 1 wherein the nutraceutical is selected from the group consisting of: curcumin, α-tocotrienol, γ-tocopherol, EGCG=epicatechin gallate, docosahexaenoic acid (DHA), resveratrol, silymarin, luteolin, magnesium, nicotinamide, riboflavin and racetams.

5. The method of claim 1, further comprising administering an antioxidant to the patient between the first and second rounds of MRS selected from a group consisting of: ascorbic acid and derivatives thereof, alpha tocopherol and derivatives thereof, rutin, quercetin, hesperedin, lycopene, resveratrol, tetrahydrocurcumin, rosmarinic acid, Ellagic acid, chlorogenic acid, oleuropein, alpha-lipoic acid, glutathione, polyphenols, and pycnogenol.

6. The method of claim 1, further comprising administering an anti-inflammatory small molecule agent to the patient between the first and second rounds of MRS.

7. The method of claim 6, wherein the anti-inflammatory small molecule is selected from the group consisting of: pioglitazone, aspirin, ibuprofen, n-acetylcysteine, and resveratrol.

8. The method of claim 1 further comprising:
(a) providing an agent with ability to inhibit host inflammatory reactions;
(b) providing an agent or therapy capable of mobilizing endogenous stem cells; and
(c) administering said agent with ability to inhibit host inflammatory reactions with said agent or therapy capable of mobilizing endogenous stem cells to the patient between the first and second rounds of MRS.

9. The method of claim 8, wherein said agent with ability to inhibit host inflammatory reactions is selected from a group consisting of: a) a small molecule, b) a nucleic acid, and c) a protein.

10. The method of claim 8, wherein said therapy capable of mobilizing endogenous stem cells is selected from a group of treatments consisting of: hyperbaric oxygen, exercise, and autohemotherapy using extracorporeal ozonation.

11. The method of claim 2, wherein laser irradiation therapy is provided transcranially in at least one wavelength, said wavelength in a range between about 620 nanometers and about 1070 nanometers.

12. The method of claim 2, wherein said laser irradiation is administered by a light source between approximately 100 .mu.W/cm2 to approximately 10 W/cm2 and a power density to the brain of the patient is administered in a non-invasive manner by transmitting light energy through the scalp and the skull to the brain, wherein the power density is at least about 0.01 mW/cm2 at a depth of approximately 2 centimeters below the dura.

13. The method of claim 11, wherein said wavelength is between about 630 nm to about 904 nm.

14. The method of claim 11, wherein said wavelength is between about 780 nm to about 840 nm.

15. The method of claim 2, wherein said laser irradiation therapy comprises placing a light source in contact with a region of skin of the patient adjacent to the brain.

16. The method of claim 12, wherein determining the surface power density of the light energy sufficient to deliver the power density of light energy to the brain consists of identifying the surface power density of the light energy sufficient for the light energy to traverse the distance between the skin surface and the brain.

17. The method of claim 3, wherein said stem cells are derived from morcelized amniotic membrane.

18. The method of claim 3, wherein said amniotic stem cells are identified based on expression of one or more antigens selected from a group consisting of: Oct-4, Rex-1, CD9, CD13, CD29, CD44, CD166, CD90, CD105, SH-3, SH-4, TRA-1-60, TRA-1-81, SSEA-4 and Sox-2.
